(19) United States
(12) Plant Patent Application Publication
(10) Pub. No.: US 2006/0206972 P1
(43) Pub. Date: Sep. 14, 2006

(54) Deciduous flowering shrub

(50) VIBUMUM SP.; VIBUMUM

(76) Inventor: W. Andrew Duvall, South Lyon, MI (US)

Correspondence Address: James M. Deimen;Suite 300, 320 N. Main Street, Ann Arbor, MI, 48104-1192, US

(21) Appl. No.: 11/074,819

(22) Filed: Mar. 8, 2005

(51) Int. Cl. A01H 5/00

(52) U.S. Cl. PLT/226

(57) ABSTRACT

A new and distinct fragrant viburnum plant named ‘Duvone’ which is distinguished by the same characteristics which make it a valuable ornamental plant; specifically, abundant spicy fragrant flowers, glossy foliage, multi-hued autumn color developing and holding after most deciduous plants have defoliated, and slow growth and tidy habit suitable for smaller scale low maintenance landscapes.

### BACKGROUND OF THE INVENTION

The present invention, a deciduous flowering shrub, relates to a new and distinct cultivar of fragrant viburnum hereinafter referred to by the cultivar name ‘Duvone.’ (Note: as used herein, “fragrant viburnum” refers to a class of viburnum characterized by their pleasant aroma, regardless of their species or cultivar.)

### DISCOVERY OF THE INVENTION

The original plant was discovered by the inventor growing in a block of Viburnum×‘Sarcoxie’ at Duvall Nursery, South Lyon, Mich. The plant is believed to be a spontaneous sport of ‘Sarcoxie’. This block of ‘Sarcoxie’ and its proceeding generation were both propagated at Duvall Nursery.

The new plant has been reproduced through numerous propagation cycles by vegetative summer softwood cuttings. Subsequent generations have remained identical to the original plant in all distinguishing characteristics.

While ‘Duvone’ more closely resembles ‘Sarcoxie’ than any other viburnum known to the inventor, it differs in three distinct aspects. (FIG. 1—‘Duvone’ is to the left of ‘Sarcoxie’. Both plants were rooted at the same time and have been grown under identical conditions.) The habit of ‘Duvone’ is rounded and compact, whereas ‘Sarcoxie’ is taller and more upright. Flowers and foliage develop two to three days earlier on ‘Duvone’ than ‘Sarcoxie.’ Finally, ‘Duvone’ flowers have a very pleasant spicy fragrance, whereas ‘Sarcoxie’ has a somewhat unpleasant fragrance.

### SUMMARY OF THE INVENTION

The following characteristics describe the ornamental value and distinguishing features of ‘Duvone’ as observed at South Lyon, Mich.
FLOWERS

Coral pink buds open to pure white flowers to abundantly complement glossy, crinkled emerging leaves (FIG. 2 and FIG. 7). The flowers have a classic spicy fragrance similar to Viburnum carlesii.
FOLIAGE

Changing foliage offers interest throughout the seasons. Young glossy, crinkled leaves mature to a lustrous green by early summer. While autumn coloration and time of color development may vary somewhat with the season, fall color is both spectacular and definitive. The plant consistently produces a multi-hued display of red, purple, orange, and yellow (FIG. 3). The plant develops its autumn color late, retaining its excellent green summer foliage as a contrast to normal autumn colors (FIG. 4). It then develops its characteristic coloration that remains after most other deciduous plants have defoliated (FIG. 5), often extending the color season until snowfall. This delayed multi-hued autumn coloration is believed to be unique among viburnum. (Locally, only Pyrus calleryana ‘Bradford’ still shows effective coloration at this time.)
HABIT

‘Duvone’ has a very neat, tidy appearance through all seasons. Under nursery culture, it has a vigorous upright habit. In a landscape situation, the growth rate slows markedly and it develops a very dense compact rounded habit (FIG. 6). This change is due to the tendency of nearly all shoots to produce only three short internodes before terminating in a flower bud. This property not only restricts the size of the plant but also enhances flowering. This characteristic is believed to be definitive.
CULTURE

Propagation by summer softwood cuttings. Culture as per other fragrant viburnums. Transplants readily balled and burlapped or from container. Sun or partial shade. Soil tolerant. Vigorous growth fosters economic nursery production.
HARDINESS

Hardy to USDA Hardiness Zone 5.
USE

Anywhere all season color and interest is desired. Foundation planting, accents, mass plantings, focal points, flowering hedges. The slow growth rate and moderate size are appropriate for low maintenance residential scale landscapes.
PESTS

None serious. Aphids and mites have been observed. Foliage appears to be disease resistant. No deer damage, either browsing or rubs, has been observed.

### BRIEF DESCRIPTION OF THE DRAWINGS

FIG. 1 is a colored photograph illustrating the differences between ‘Duvone’ on the left and Viburnum×‘Sarcoxie’ on the right;

FIG. 2 is a colored photograph illustrating ‘Duvone’ in flower;

FIG. 3 is a colored photograph illustrating fall coloration;

FIG. 4 is a colored photograph illustrating retention of summer foliage concurrent with “normal” deciduous fall color. ‘Duvone’ is in lower left foreground. Photograph taken Oct. 12, 2003;

FIG. 5 is a colored photograph illustrating effective fall color of ‘Duvone’ remaining after other deciduous plants have defoliated. Photograph taken Nov. 14, 2003;

FIG. 6 is a winter photograph showing the habit and branching character; and

FIG. 7 is a close-up photograph showing the buds, flowers and adjacent foliage.

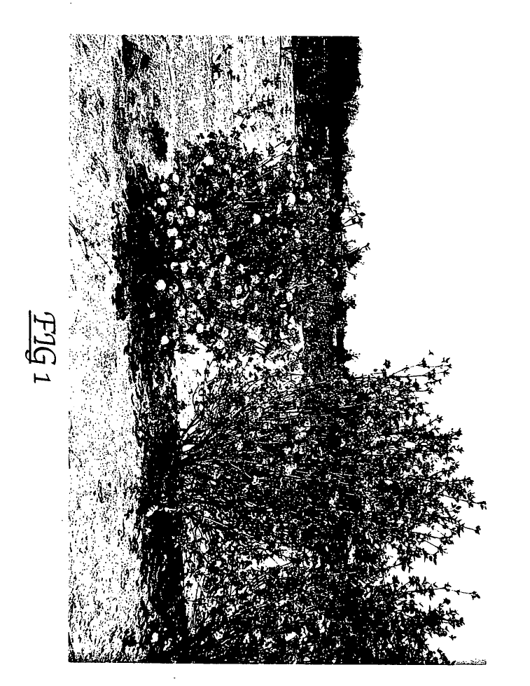

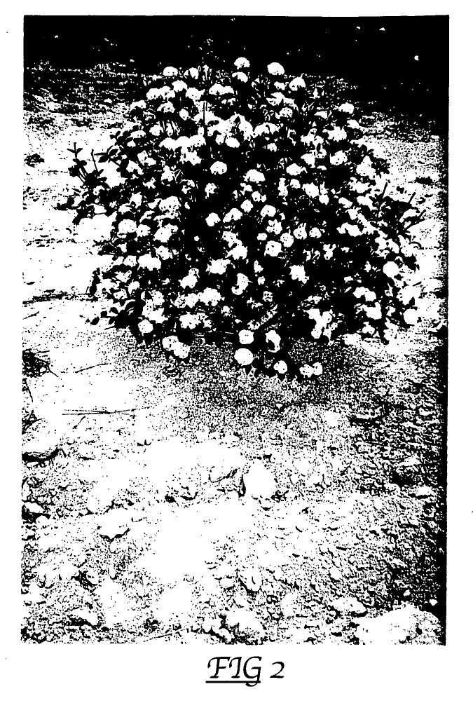

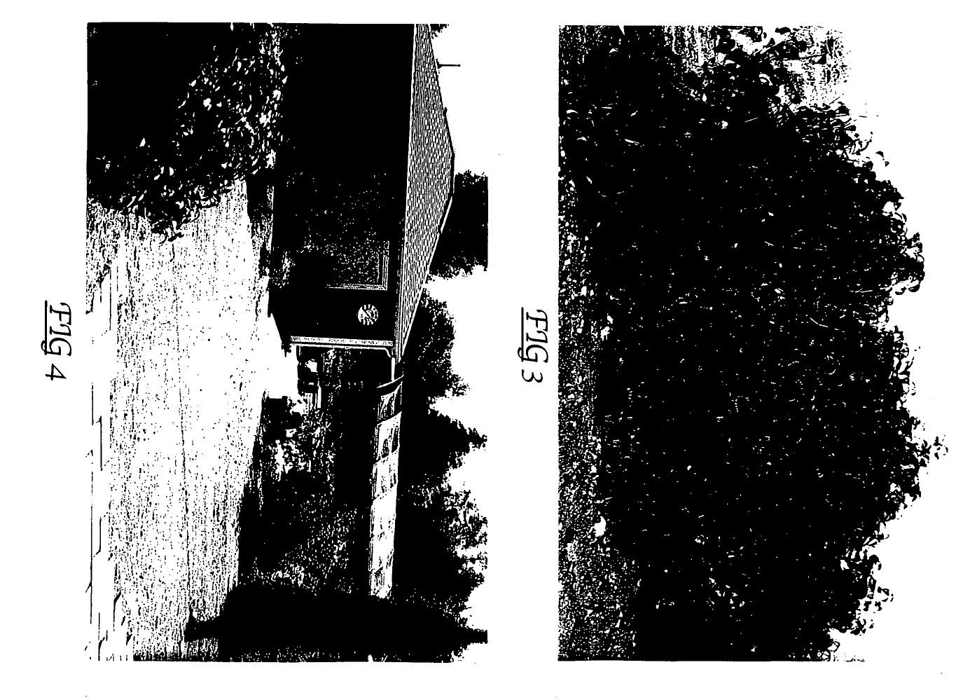

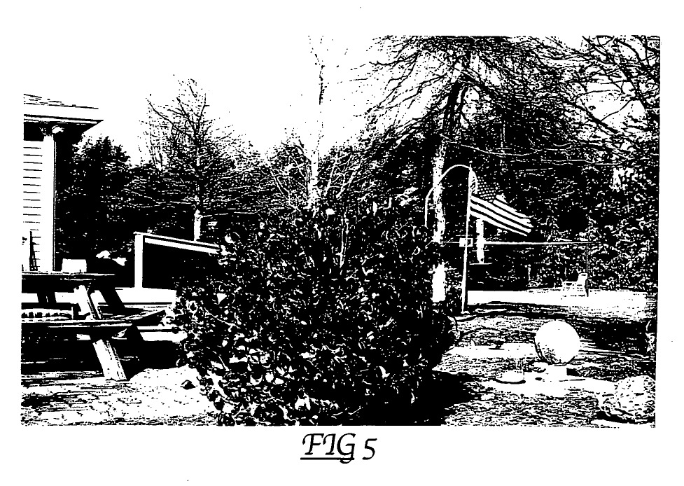

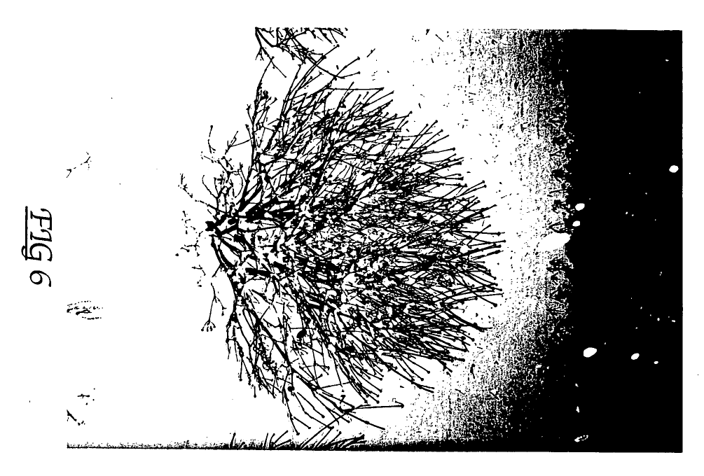

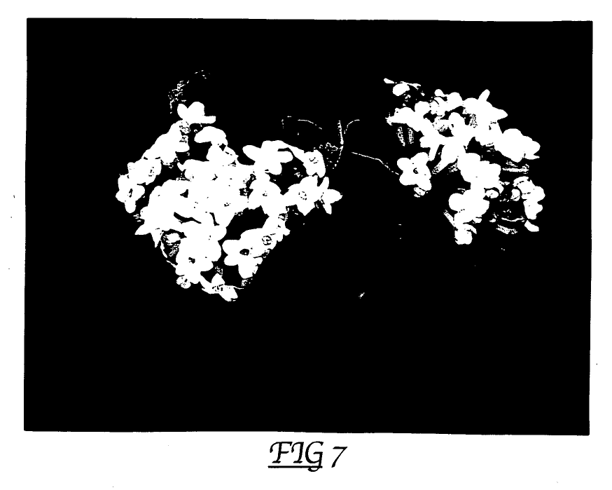

### DESCRIPTION OF THE PREFERRED EMBODIMENTS

The following is a detailed description of my new and distinct variety of fragrant viburnum cultivar ‘Duvone’ discovered and grown outdoors at South Lyon, Mich. Where possible, reference is made to specific colors on the chart issued by The Royal Horticultural Society, London, England.
Botanical classification: Viburnum×burkwoodii ‘Duvone.’
Parentage: Believed to be a naturally occurring sport of Viburnum×burkwoodii ‘Sarcoxie,’ not patented, at Duvall Nursery.
   Form/size.—Compact rounded deciduous shrub. Original plant propagated as a cutting in 1992 is now 1.3 m wide by 1.2 m tall. This plant was transplanted in 1997, sheared in 1999, again transplanted in 2002.
   Growth Rate.—Young plants under nursery conditions vigorous and upright, annual growth 30 to 45 cm per year; mature plants rounded and slow growing, annual growth 13 to 16 cm per year.
   Hardiness.—Hardy in USDA Hardiness Zone 5. Survived the winter of 1993-94 with 10 consecutive days of minus 20 degrees F. low temperatures and high temperature never reaching 0 degrees F. with no snow cover as a first year field transplant, then undiscovered.
   Twigs and stems.—Branches of current year 3-5 mm wide, stiff, ascending, densely stellate, Greyed-Orange Group 176 B; internodes mostly 2-10 cm long, except on rapidly growing shoots; older twigs Greyed-Brown Group 199A, becoming glabrous, bark irregularly low ridged; vegetative winter buds naked, lanceolate, densely stallate, 4-14 mm long, Greyed-Orange Group 177A. Dormant flower bud plus or minus hemispherical, 8-10 mm wide, 5-7 mm high.
   Leaves.—Opposite, simple, petioles 4-10 (−15) mm long; blades coriaceous, broadly elliptic to very broadly ovate, (3-) 5-9 cm long, (2-) 3-7 cm wide, the apex acute, the base broadly cuneate to rounded or even subcordate on the largest leaves, the margins shallowly and irregularly dentate with 2-4 teeth/cm, becoming plus or minus entire near apex; pinnately 3-5 veined per side; upper surface, Green Group 137B, strongly rugose with impressed veins when young, sparsely pale brown stellate pubescent, glabrescent and plus or minus glossy at maturity; undersurface Yellow-Green Group 147C, more densely reddish-brown stellate-pubescent even at maturity, veins raised beneath. Leaves persistent very late into fall, eventually developing fall color and turning deep red, Red Group 46B, with strong purple, Purple Group 79A, yellow, Yellow Group 10B, and orange, Orange Group 28A, overtones.
   Flowers.—Inforescences terminal, cymose, broadly rounded, 4.5-5 cm wide, 2.5-3 cm high, many flowered (average 67), short pedunculate, the peduncle stellate pubescent; pedicels with a small, stellate-pubescent, papery bract. Flowers 5-merous, regular, perfect, 9-10 mm in diameter, pleasantly spicy-fragrant; Calyx lobes minute triangular; corolla reddish in bud, Red Group 49D, pure white when open, lobes spreading; Stamens 5, attached near the base of the corolla tube, anthers yellow.
   Fruit.—Drupe, oblong-ellipsoid, slightly beaked, ca. 10-13 mm long, 7-9 mm wide, 3 mm thick, black at maturity; stone 8-9 mm long, ca 6 mm wide, ellipsiod, strongly flattened, shallowly ridged on both faces; ultimate pedicles finely stellate-pubescent.

1. A new and distinct viburnum plant originating as a sport of Viburnum×burkwoodii ‘Sarcoxie’ as herein illustrated and described, a shrub characterized by abundant fragrant flowers, glossy foliage, late fall color and compact habit.
